(19) United States
(12) Patent Application Publication
(10) Pub. No.: US 2026/0224652 A1
(43) Pub. Date: Aug. 6, 2026

(54) ENRICHED PELARGONIUM GRAVEOLENS EXTRACTS FOR THE TREATMENT OF TRAUMATIC BRAIN INJURIES

(71) Applicants: THE STATE OF ISRAEL, MINISTRY OF AGRICULTURE & RURAL DEVELOPMENT, AGRICULTURAL RESEARCH, Rishon Lezion (IL); SHEBA IMPACT LTD., Ramat-Gan (IL)

(72) Inventors: Anat ELMANN, Rehovot (IL); Sigal LIRAZ ZALTSMAN, Bnei-Atarot (IL); Nativ DUDAI, Kfar Yehezkel (IL)

(21) Appl. No.: 19/149,361

(22) PCT Filed: Jan. 14, 2024
(86) PCT No.: PCT/IL2024/050049

§ 371 Date: Jul. 18, 2025

Related U.S. Application Data
(60) Provisional application No. 63/480,570, filed on Jan. 19, 2023.

(51) Int. Cl. A61K 36/185; A61K 9/00; A61K 31/047; A61K 31/11; A61K 31/122; A61P 25/00

(52) U.S. Cl. CPC A61K 36/185; A61K 9/0019; A61K 9/0043; A61K 9/0053; A61K 31/047; A61K 31/11; A61K 31/122; A61P 25/00

(57) ABSTRACT

The invention concerns methods and compositions for treating, preventing, or ameliorating traumatic brain injury (TBI), and for improving cognitive function of a subject suffering from TBI, comprising essential oil extracted from Pelargonium graveolens (Pg oil) enriched with a monoterpene or monoterpenoid.

### TECHNOLOGICAL FIELD

The present disclosure generally relates to a novel treatment for brain damage.

### BACKGROUND ART

References considered to be relevant as background to the presently disclosed subject matter are listed below:
1. Lifshitz J. Experimental CNS Trauma: A General Overview of Neurotrauma Research. In: Kobeissy F H, ed. Brain Neurotrauma: Molecular, Neuropsychological, and Rehabilitation Aspects. Boca Raton (FL)2015.
2. Maas, A. I., et al., Collaborative European NeuroTrauma Effectiveness Research in Traumatic Brain Injury (CENTER-TBI): a prospective longitudinal observational study. Neurosurgery, 2015. 76(1): p. 67-80.
3. Maas, A. I. R., et al., Traumatic brain injury: integrated approaches to improve prevention, clinical care, and research. Lancet Neurol, 2017. 16(12): p. 987-1048.
4. Gardner R C, Burke J F, Nettiksimmons J, Kaup A, Barnes D E, Yaffe K. Dementia risk after traumatic brain injury vs nonbrain trauma: the role of age and severity. JAMA neurology. December 2014; 71(12):1490-1497.
5. Nordstrom P, Michaelsson K, Gustafson Y, Nordstrom A. Traumatic brain injury and young onset dementia: a nationwide cohort study. Annals of neurology. March 2014; 75(3):374-381.
6. Beauchamp K, Mutlak H, Smith W R, Shohami E, Stahel P F. Pharmacology of traumatic brain injury: where is the “golden bullet”? Molecular medicine. November-December 2008; 14(11-12):731-74.0.
7. Narayan R K, Michel M E, Ansell B, et al. Clinical trials in head injury. Journal of Neurotrauma. May 2002; 19(5):503-557.
8. Abbasloo E, Dehghan F, Khaksari M, et al. The anti-inflammatory properties of Satureja khuzistanica Jamzad essential oil attenuate the effects of traumatic brain injuries in rats. Scientific reports. Aug. 18 2016; 6:31866.
9. Elmann, A., Mordechay, S., Rindner, M., and Ravid, U. (2010). Anti-neuroinflammatory effects of the essential oil from Pelargonium graveolens in microglial cells. Journal of Functional foods. 2: 17-22.
10. WO 2013/168090
11. Ravid U. and Putievsky E. (1984). The influence of harvest dates and leaf location on the essential oil content and major components of Pelargonium Graveolens L. Acta Horticulturae 144:159-165.
12. Putievsky E, Ravid U and Dudai N (1990). The effect of water stress on yield components and essential oil of Pelargonium Graveolens L. J. Ess. Oil Res., 2:111-114.
13. Chen Y, Constantini S, Trembovler V, Weinstock M, Shohami E. An experimental model of closed head injury in mice: pathophysiology, histopathology, and cognitive deficits. Journal of neurotrauma. October 1996; 13(10):557-568.
14. Flierl M A, Stahel P F, Beauchamp K M, Morgan S J, Smith W R, Shohami E. Mouse closed head injury model induced by a weight-drop device. Nature protocols. 2009; 4(9):1328-1337.
15. Beni-Adani, L., Gozes, I., Cohen, Y., Assaf, Y., Steingart, R. A., Brenneman, D. E., Eizenberg, O., Trembolver, V., Shohami, E., 2001. A peptide derived from activity-dependent neuroprotective protein (ADNP) ameliorates injury response in closed head injury in mice. J Pharmacol Exp Ther 296, 57-63.
16. Tsenter J, Beni-Adani L, Assaf Y, Alexandrovich A G, Trembovler V, Shohami E., 2008. Dynamic changes in the recovery after traumatic brain injury in mice: effect of injury severity on T2-weighted MRI abnormalities, and motor and cognitive functions. J Neurotrauma 25(4):324-33.

Acknowledgement of the above references herein is not to be inferred as meaning that these are in any way relevant to the patentability of the presently disclosed subject matter.

### BACKGROUND

Traumatic brain injury (TBI) affects millions of people each year, worldwide and is a major cause of death and disability among young people [1]. The World Health Organization has declared TBI a significant public health problem, and the US Department of Defense (DoD) has reported that more than 313,816 service personnel suffer brain damage [2, 3]. A traumatic head injury causes immediate symptoms as well as chronic symptoms that may last for many years after the injury among them, cognitive decline manifested in decreased ability to learn, remember, and concentrate, and the onset of dementia at a relatively young age [4, 5].

There is currently no effective pharmacological treatment for both immediate and long-term effects and cognitive decline. These consequences harm the quality of life of the victim, his family and constitute a great economic burden on the health system, and the caring family [6, 7].

Abbasloo et al [8] disclose that intraperitoneal injection of Satureja khuzistanica Jamzad essential oil attenuates the effects of traumatic brain injuries in rats.

The essential oil extracted from the plant Pelargonium graveolens was previously shown to inhibit activation of microglial cells in culture [9]. WO 2013/168090 [10] discloses that a Pelargonium graveolens constituent (i.e. (S) (−) citronellol) inhibits in vitro the activity of the enzyme acetyl choline esterase. In addition, Pelargonium graveolens oil (also termed “geranium oil”) was shown to be effective in ameliorating various physiological parameters of Parkinson's disease in an animal model of the disease (MPTP).

There is an urgent need to develop a treatment that can slow down or eliminate the late onset symptoms and the cognitive decline associated with TBI and thereby to reduce the vast personal, social, and economic burden of these debilitating conditions.

### GENERAL DESCRIPTION

In a first aspect, the present invention provides a pharmaceutical or nutritional composition for treating, preventing, or ameliorating traumatic brain injury in a subject, the composition comprising:
(a) essential oil extracted from Pelargonium graveolens (Pg oil);
(b) at least one additional monoterpene or monoterpenoid, wherein the final concentration (weight/weight) of said at least one additional monoterpene or monoterpenoid is at least 50% higher than the concentration of said monoterpene or monoterpenoid in the Pg oil; and optionally
(c) one or more physiologically acceptable carriers.

In another aspect, the present invention provides a pharmaceutical or nutritional composition for improving cognitive function of a subject suffering from traumatic brain injury, the composition comprising:
(a) essential oil extracted from Pelargonium graveolens (Pg oil);
(b) at least one additional monoterpene or monoterpenoid, wherein the final concentration (weight/weight) of said at least one additional monoterpene or monoterpenoid is at least 50% higher than the concentration of said monoterpene or monoterpenoid in the Pg oil; and optionally
(c) one or more physiologically acceptable carriers.

In another aspect, the present invention provides a pharmaceutical or nutritional composition for treating, preventing, or ameliorating traumatic brain injury in a subject, the composition comprising:
(a) essential oil extracted from Pelargonium graveolens (Pg oil);
(b) at least one exogenous monoterpene or monoterpenoid; and optionally one or more physiologically acceptable carriers.

In another aspect, the present invention provides a composition comprising:
(a) essential oil extracted from Pelargonium graveolens (Pg oil);
(b) at least one additional monoterpene or monoterpenoid, wherein the final concentration in the composition (weight/weight) of said at least one additional monoterpene or monoterpenoid is at least 50% higher than the concentration of said monoterpene or monoterpenoid in the Pg oil; and optionally
(c) one or more physiologically acceptable carriers
for use in a method of treating, preventing, or ameliorating TBI in a subject in need thereof, the method comprising administering to the subject a therapeutically effective amount of said composition.

In another aspect, the present invention provides a composition comprising:
(a) essential oil extracted from Pelargonium graveolens (Pg oil);
(b) at least one additional monoterpene or monoterpenoid, wherein the final concentration in the composition (weight/weight) of said at least one additional monoterpene or monoterpenoid is at least 50% higher than the concentration of said monoterpene or monoterpenoid in the Pg oil; and optionally
(c) one or more physiologically acceptable carriers
for use in a method of improving cognitive function of a subject suffering from traumatic brain injury, the method comprising administering to the subject a therapeutically effective amount of said composition.

In another aspect, the present invention provides a method of treating, preventing, or ameliorating TBI, the method comprising administering to a subject in need thereof a therapeutically effective amount of a composition comprising:
(a) essential oil extracted from Pelargonium graveolens (Pg oil);
(b) at least one additional monoterpene or monoterpenoid, wherein the final concentration in the composition (weight/weight) of said at least one additional monoterpene or monoterpenoid is at least 50% higher than the concentration of said monoterpene or monoterpenoid in the Pg oil; and optionally
(c) one or more physiologically acceptable carriers.

In another aspect, the present invention provides a method of improving cognitive function of a subject suffering from traumatic brain injury, the method comprising administering to said subject a therapeutically effective amount of a composition comprising:
(a) essential oil extracted from Pelargonium graveolens (Pg oil);
(b) at least one additional monoterpene or monoterpenoid, wherein the final concentration in the composition (weight/weight) of said at least one additional monoterpene or monoterpenoid is at least 50% higher than the concentration of said monoterpene or monoterpenoid in the Pg oil; and optionally
(c) one or more physiologically acceptable carriers.

In an embodiment, said cognitive function is at least one of learning ability, or memory (e.g., spatial memory).

In an embodiment, the TBI is caused by an external force, rapid acceleration, blast waves, or penetration of the skull that reaches brain tissue.

In an embodiment, said subject is a human.

In an embodiment, said subject is an animal selected from the group consisting of fish, sheep, pigs, cattle, goats, horses, camels, buffalo, rabbits, cats, dogs, and primates.

In an embodiment, said composition is in the form of a food article, a beverage, a food additive, food supplement, botanical drug, or a pharmaceutical composition.

In an embodiment, said composition is in the form of tablets, capsules, liquid syrups, oils, nasal spray, nasal drops, soft gels, suppositories, patches, and enemas.

In an embodiment, said composition is a pharmaceutical composition comprising a pharmaceutically acceptable carrier.

In an embodiment, said pharmaceutical composition is administered by oral, intraperitoneal, subcutaneous, transcutaneous, topical, intramuscular, intraarticular, subconjunctival, intranasal, or intraocular administration.

In an embodiment, said at least one additional monoterpene or monoterpenoid is selected from a group consisting of citronellol, citronellyl formate, linalool, geraniol and menthone.

In an embodiment, said composition comprises a concentration of between about 30% and 80% citronellol.

In an embodiment, said composition comprises a concentration of between about 10% and 80% citronellyl formate.

In an embodiment, said composition is administered at a concentration of between about 0.5 mg/kg and 50 mg/kg.

In an embodiment, said composition comprises less than 200 mg/kg essential oil extracted from Pelargonium graveolens.

In an embodiment, said composition is administered once, twice, three times or more daily, or once every two days or every three days for a treatment session of between about 5 days and four weeks, or more following the TBI, or administered in a prophylactic manner to subjects at risk of TBI.

In an embodiment, said composition is administered in a treatment session that comprises between about 8 and about 12 daily treatments, e.g., 9 daily treatments per session.

In an embodiment, said composition is administered in a treatment session that comprises between about 8 and about 12 daily treatments, e.g., 9 daily treatments per session, followed by chronic administration once a week.

In an embodiment, at least one additional treatment session with said composition is administered to said subject between about 4 months, 5 months, 6 months, 7 months, 8 months, 9 months, 10 months, 11 months, 12 months, or more following the TBI.

In an embodiment, said composition is provided orally at a concentration of between about 5 mg/kg and about 50 mg/kg once a day or once every two or three days for a treatment session that comprises between about 8 and about 12 daily treatments per session, e.g., 9 daily treatments per session.

In an embodiment, said composition is provided by intranasal administration at a concentration of between about 0.5 mg/kg and about 5 mg/kg once a day or once every two or three days for a treatment session that comprises between about 8 and about 12 daily treatments per session, e.g., 9 daily treatments per session.

In an embodiment, said composition is provided by inhalation at a concentration of between about 0.1% and about 10% once a day or once every two or three days for a treatment session that comprises between about 8 and about 12 daily treatments per session, e.g., 9 daily treatments per session.

In an embodiment, one additional treatment session of two weeks is provided to said subject 9 months following the TBI.

In an embodiment, the composition is a sustained release composition.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIG. 1 is a scheme showing the study design of the CHI model and the behavioral/cognitive tests.

FIG. 2A is a graph showing the neurological severity score (NSS) values for each experimental group, measured at different time points after the traumatic brain injury (Time post TBI (days)).

FIG. 2B is a graph showing the same results as FIG. 2A in a bar format showing the relative significance of each of the tested groups. [*] indicates a P-value of <0.05, [**] indicates a P-value of <0.005, [***] indicates a P-value of <0.0008, [****] indicates a P-value of <0.0001, by repeated measure 2-way ANOVA.

FIG. 3 is a graph showing latency to escape box in seconds (sec) in a Barnes maze (BM) test performed two weeks after a closed head injury (CHI), which is a model system designed to study traumatic brain injury (TBI). The mice were tested 1 day, 2 days, 3 days, and 4 days (defined as the acquisition period) after completion of the two weeks treatment. This test measures the learning ability. The following groups were tested: Sham, CHI+vehicle, CHI+Citronellyl formate 80 mg/kg, CHI+Citronellol 25 mg/kg, CHI+100 mg/kg Pg oil, CHI+100 mg/kg Pg oil+Citronellol 25 mg/kg, and CHI+100 mg/kg Pg oil+Citronellol 25 mg/kg+Citronellyl formate 80 mg/kg.

FIG. 4 is a graph showing the same results as FIG. 3 in a bar format showing the relative significance of each of the tested groups. Results are presented as mean±SD [*] indicates a P-value of <0.05, [**] indicates a P-value of P<0.005, [***] indicates a P-value of P<0.0005, [****] indicates a P-value of P<0.0001, ns=not significant, by Mixed-effects analysis using GraphPad Prism 9.5.0.

FIG. 5 is a graph showing results of a Novel Object Recognition (NOR) test. Results are presented as percentage of the time spent by a novel object out of the total time. The following groups were tested: Sham, CHI+vehicle, CHI+Citronellyl formate 80 mg/kg, CHI+Citronellol 25 mg/kg, CHI+100 mg/kg Pg oil, CHI+100 mg/kg Pg oil+Citronellol 25 mg/kg, and CHI+100 mg/kg Pg oil+Citronellol 25 mg/kg+Citronellyl formate 80 mg/kg. Rounds-3. Results are presented as mean±SD [**] indicates a P-value of 0.005, [***] indicates a P-value of <0.0002, and ns=not significant, by Mixed-effects analysis using GraphPad Prism 9.5.0.

FIG. 6A is a graph showing the preference index (PI) values as measured in a Y maze test for each experimental group. Each dot represents a tested mouse, and the graph shows the distribution of mice and the median value (represented by a solid line). The dotted line represents PI=0. [**] indicates a P-value of 0.002 by one way ANOVA.

FIG. 6B is a graph showing the percent of the mice (% animals) with spatial memory and with an impaired spatial memory in each of the experimental groups shown in FIG. 6A.

### DETAILED DESCRIPTION OF EMBODIMENTS

The present invention is based on the surprising finding that feeding mice after a closed head injury (an animal model of TBI) with an essential oil extracted from the plant Pelargonium graveolens (Pg) enriched with monoterpenes or monoterpenoids (e.g., citronellol and/or citronellyl formate) significantly improved their learning and memory performance as evidenced in behavioral tests.

Prior experiments revealed that a concentration of 200 mg/kg of the Pg oil is active, but is less effective than a concentration of 500 mg/kg Pg oil in improving learning and memory in mice suffering from CHI. As shown in Example 1 below, the Pg oil comprises many compounds, including among others citronellol and citronellyl formate. Surprisingly, an enriched composition comprising a suboptimal concentration of Pg oil (e.g., a composition comprising 100 mg/kg Pg oil) supplemented with citronellol and optionally also with citronellyl formate, was found to have a beneficial effect on learning and memory as measured in various behavioral tests including Barnes maze (BM), novel object recognition (NOR) and Y maze. The treated mice performed significantly better than those treated with the Pg oil alone. Namely, supplementing the Pg oil with citronellol and optionally also with citronellyl formate largely enhanced its efficiency.

Interestingly, a significant reduction in the neurological severity score (NSS), which is an indication of improvement mostly in motor functions, was observed in all the experimental groups (namely also in mice that were treated with a suboptimal concentration of the Pg oil as a single agent, or citronellol or citronellyl formate as single agents). This observation emphasizes the unique effect of the combination treatment on the cognitive/behavioral functions of the traumatized mice.

Therefore, the present invention concerns a pharmaceutical or nutritional composition for treating, preventing, or ameliorating traumatic brain injury in a subject, the composition comprising essential oil extracted from Pelargonium graveolens with an enriched monoterpene or monoterpenoid content, and one or more physiologically acceptable carriers.

In a first of its aspects, the present invention provides a pharmaceutical or nutritional composition for treating, preventing, or ameliorating traumatic brain injury in a subject, the composition comprising:
(a) essential oil extracted from Pelargonium graveolens (Pg oil);
(b) at least one additional monoterpene or monoterpenoid, wherein the final concentration (weight/weight) of said at least one additional monoterpene or monoterpenoid is at least 50% higher than the concentration of said monoterpene or monoterpenoid in the Pg oil; and optionally
(c) one or more physiologically acceptable carriers.

In another aspect, the present invention provides a pharmaceutical or nutritional composition for treating, preventing, or ameliorating traumatic brain injury in a subject, the composition comprising:
(a) essential oil extracted from Pelargonium graveolens (Pg oil);
(b) at least one exogenous monoterpene or monoterpenoid; and optionally
(c) one or more physiologically acceptable carriers.

As used herein the term “exogenous” refers to a monoterpene or monoterpenoid which is not naturally present in the Pg oil.

In an embodiment, the composition of the invention comprises a suboptimal concentration of Pg oil. As used herein the term “suboptimal” refers to a concentration of Pg oil which, when used alone, has a reduced functional activity, or is ineffective, as measured for example, using cognitive/behavioral tests. In some embodiments a suboptimal concentration of Pg oil is a concentration lower than 200 mg/kg, e.g., 100 mg/kg. Accordingly, in an embodiment, the composition of the invention comprises less than 200 mg/kg Pg oil, e.g., 100 mg/kg Pg oil. Namely, the enrichment of the composition with the monoterpene or monoterpenoid (e.g., citronellol or citronellyl formate) compensates for the reduced concentration of the Pg oil and restores its activity.

As used herein the term “pharmaceutical composition” refers to a composition comprising Pelargonium graveolens (Pg) essential oil enriched with at least one additional monoterpene or monoterpenoid, wherein said composition is provided as a medicament. As used herein the term “nutritional composition” refers to a composition comprising Pelargonium graveolens (Pg) essential oil enriched with at least one additional monoterpene or monoterpenoid, wherein said composition is provided as a food supplement, nutrition additive, food, or beverage.

The terms “Pelargonium graveolens (Pg) essential oil” or “Pelargonium graveolens (Pg) oil” (also known as “geranium oil”) are used interchangeably herein and refer to an extract of the Pelargonium graveolens (Pg) plant foliage. The essential oil can be extracted from the plant foliage using any method known in the art, for example using a steam or hydro-distillation apparatus (see e.g., Elmann et al [9] or [10] which is incorporated herein by reference), or cold press. A non-limiting example of a distillation apparatus is a hydro-distillation Clevenger apparatus system.

The essential oil extracted from the plant Pelargonium graveolens is a defined mixture of phytochemicals [see for example 11, 12]. In one embodiment, the content of the essential oil can be identified by Gas Chromatography-Mass Spectrometry (GC-MS). Non-limiting examples of the essential oil content are provided in Example 1, Table 1. It is characterized by low molecular weight molecules (~150). Due to this low molecular weight and lipophilicity of the essential oil components, they can cross the blood-brain barrier and affect processes and cells in the brain.

The essential oil of Pg is defined by the FDA as GRAS (generally recognized as safe) and therefore it is safe for consumption and use in the food industry.

As used herein the term “enriched” refers to a high content of one or more monoterpenes or monoterpenoids in the composition. In one embodiment, the monoterpene or monoterpenoid is originally present in the Pg oil and its natural amount is increased. In such case, a high content of one or more monoterpenes or monoterpenoids is defined for example as a concentration of said one or more monoterpenes or monoterpenoids that is at least 50% higher than its concentration in a wildtype Pg plant. Such higher levels of monoterpenes or monoterpenoids can be achieved by endogenic or exogenic means. For example, an endogenic enrichment can be achieved by enhancing monoterpene or monoterpenoid production by the plant using a genetic manipulation (e.g., by recombinant expression) or using cross breeding of different plant species to increase the monoterpene or monoterpenoid production. Exogenic enrichment is performed by inclusion of additional monoterpenes or monoterpenoids in the composition thereby increasing their relative concentration. In another embodiment, the monoterpene or monoterpenoid is an exogenous monoterpene or monoterpenoid which is not originally present in the Pg oil, and which is added in an amount which constitutes at least 15% of the composition.

As used herein, the terms “monoterpene” and “monoterpenoid” also encompass acetate and formate derivatives thereof.

Non-limiting examples of a monoterpene or monoterpenoid include: citronellol, citronellyl formate, linalool, geraniol and menthone.

In some embodiments, the enriched composition of the invention comprises a concentration of between about 30% and 80% citronellol.

In some embodiments, the enriched composition of the invention comprises a concentration of between about 10% and 80% citronellyl formate.

The terms “treating”, “preventing”, or “ameliorating” is used conventionally and refers to the management or care of a subject for the purpose of combating, alleviating, reducing, relieving, or improving a subject's traumatic brain injury, or any symptom thereof. The term also encompasses prophylactic treatment of subjects at risk of TBI. The terms encompass any reduction in the subject's traumatic brain injury, or any symptom thereof as evidenced, for example, by measurement of cognitive, physiological, radiological (e.g., by Magnetic Resonance Imaging (MRI)), and/or clinical indicators of brain injury.

As used herein the term “Traumatic Brain Injury (TBI)” refers to a disruption in the normal function of the brain that is caused by an external force such as a direct impact on the head (blow, bump, or jolt), rapid acceleration, blast waves, or penetration of the skull that reaches brain tissue. TBI may be associated with characteristic cognitive, emotional, physical, social, and behavioral symptoms indicating alteration in the normal brain function: loss of or decreased consciousness, loss of memory (amnesia), focal neurological deficits such as muscle weakness, loss of vision, change in speech, and/or alteration in mental state such as disorientation, slow thinking, difficulty in concentrating, sleep disorders, anxiety, mood disorders and/or social interaction.

The insult severity of TBI can be mild (e.g., a concussion), moderate, or severe, depending on the extent of damage to the brain. Mild cases may result in a brief change in mental state or consciousness followed by complete recovery, although 20% of subjects suffering from mild TBI sustain chronic/long term problems. Severe cases may result in permanent disability, extended periods of unconsciousness, coma, or even death.

As shown in the Examples below treatment of mice suffering from closed head injury (as a model for traumatic brain injury) with the enriched Pg oil of the invention (Pg oil enriched with citronellol and optionally also with citronellyl formate), resulted in remarkable improvement of cognitive functions after the trauma.

Accordingly, the present invention also provides a pharmaceutical or nutritional composition for improving cognitive function of a subject suffering from traumatic brain injury, the composition comprising:
(a) essential oil extracted from Pelargonium graveolens (Pg oil);
(b) at least one additional monoterpene or monoterpenoid, wherein the final concentration (weight/weight) of said at least one additional monoterpene or monoterpenoid is at least 50% higher than the concentration of said monoterpene or monoterpenoid in the Pg oil; and optionally
(c) one or more physiologically acceptable carriers.

It should be noted that the composition of the invention also improves motor functions following TBI.

As used herein the term “improving” refers to any amelioration, elimination, remedy or slowing down of the cognitive decline which occurs in a subject suffering from TBI. Improvement can be manifested, for example in an increased ability to learn, remember, or concentrate. Multiple tests are known in the literature for assessing the cognitive functions of subjects suffering from TBI. Any of these tests may be employed to assess the improvement in the subject's cognitive state or functions, and it is in the physician's discretion to select the most appropriate tool. Non-limiting examples include the MoCA (Montreal Cognitive Assessment) test, which is mostly used for detecting mild cognitive impairment, the MMSE (mini-mental state examination) test which is mostly used for finding more serious cognitive impairment, Mini-Cog evaluation, and others.

The compositions of the invention may be administered as such, or may be incorporated into food products, beverages (e.g., juices, tea) or combined with commonly used food additives such as corn syrup.

In one embodiment, the composition of the invention is a nutritional composition administered by feeding or drinking.

In other embodiments, the composition of the invention is a pharmaceutical composition that can be administered and dosed in accordance with good medical practice. For example, the pharmaceutical composition can be introduced to the body by any suitable route including oral, intraperitoneal, subcutaneous, transcutaneous, topical, intramuscular, intraarticular, subconjunctival, or mucosal, e.g., intranasal, or intraocular administration. The compositions may be administered systemically or may be locally administered. Local administration may be facilitated by using an implant that acts to retain the active dose at the site of implantation. The active agent may be formulated for immediate activity, or it may be formulated for sustained release.

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, formulations are prepared by uniformly and intimately bringing into association the active ingredients of the invention with liquid carriers or finely divided solid carriers or both, and then, if necessary, shaping the product.

The compositions may be formulated into any of many possible dosage forms such as, but not limited to, tablets, capsules, liquid syrups, oils, nasal spray, nasal drops, soft gels, suppositories, patches, and enemas.

The Pg oil extract in the composition may be diluted to a desired final concentration by mixing with an appropriate vehicle solution, e.g., an oily solution, including but not limited to any plant-derived essential oil, plant-derived oil, or medium chain triglycerides (MCT). In one embodiment the Pg oil is diluted in olive oil. As used in the Examples section below, the term “vehicle” refers to olive oil given to the animals as a control without the active agent.

In addition to the ingredients particularly mentioned above, the compositions of the invention may also include other agents conventional in the art having regard to the type of composition in question.

Still further, the compositions of the invention and any components thereof may be applied as a single daily dose or multiple daily doses.

The compositions may be administered for a treatment session of between about 5 days to about four weeks or more immediately after the trauma. In one embodiment, the compositions are administered daily or once every two or three days for a period of 10 to 14 days immediately after trauma. In one embodiment, a total number of between 5 treatments and 15 treatments is provided following the trauma starting immediately after trauma (namely within minutes or hours), or one day after the trauma. A person skilled in the art would realize that the sooner the first treatment is given, the better. Therefore, in specific embodiments a total number of 5, or 6, or 7, or 8, or 9, or 10, or 11, or 12, or 13, or 14, or 15, or more daily treatments are provided to the subject. In a specific embodiment the treatment session comprises between 8 and 12 daily treatments. In another specific embodiment the treatment session comprises 9 daily treatments. In some embodiments, one or more additional treatment sessions may be given to the subjects between about one month and about one year after the trauma. In one embodiment, the additional treatment session comprises between 8 and 12 daily treatments given over a period of about two weeks. In one embodiment, the additional treatment session is given 9 months after the injury and comprises about 9 daily treatment doses given over a period of about two weeks. In one embodiment, the “daily treatment” refers to one administration of the composition per day. In other embodiments, the “daily treatment” refers to two or three administrations of the composition per day.

In another embodiment, the compositions are administered as a prophylactic treatment for subjects at risk of head trauma or TBI, e.g., soldiers, policemen, fire fighters, or athletes. In a specific embodiment, the subject receives between 1 and 5 daily treatments commencing several days prior to the intended risky event. In one embodiment, the subject receives a total amount of three daily treatments commencing two days prior to the risky event.

In another embodiment, the compositions are administered for an unlimited time-period (also referred to as chronic administration), for example for one year, two years or more, or as a lifetime treatment. In one embodiment, after the initial intensive treatment session, the compositions may be given once a week for as long as desired (e.g., for one year, two years, or more, or as a lifetime treatment), also referred to herein as “chronic treatment”. For example, the compositions may be given as a food supplement for regular consumption.

The composition of the invention can be administered alone, or in combination with other active agent(s). In certain embodiments the additional active agents include, but are not limited to, 3,5,4′-trihydroxy-6,7,3′-trimethoxyflavone (TTF), or steroids.

The compositions of the present invention may be combined with other types of treatments including behavioral therapy, rehabilitation therapy, diet restrictions and pharmacological intervention.

In another aspect, the present invention provides a method of treating, preventing, or ameliorating TBI, the method comprising administering to a subject in need thereof a therapeutically effective amount of a composition comprising:
(a) essential oil extracted from Pelargonium graveolens (Pg oil);
(b) at least one additional monoterpene or monoterpenoid, wherein the final concentration in the composition (weight/weight) of said at least one additional monoterpene or monoterpenoid is at least 50% higher than the concentration of said monoterpene or monoterpenoid in the Pg oil; and optionally
(c) one or more physiologically acceptable carriers.

By the term “administering”, it is meant that the composition is delivered to a subject by any means or route which is effective to achieve the desired result, including e.g., oral, parenteral, enteral, intraperitoneal, topical, transdermal (e.g., using any standard patch), subcutaneous, intravenous, intra-arterial, intramuscular, buccal, sublingual, ophthalmic, nasal, by aerosol, by inhalation, rectal, vaginal, and intrathecal.

In some embodiments the amount of the Pg oil in the composition is between about 0.5 mg/kg to 50 mg/kg per day or once every two or three days, depending upon the subject's physical condition, the severity of disease, mode of administration, etc. In an embodiment, the amount of the Pg oil in the composition is less than 16 mg/kg. In specific embodiments the composition of the invention is orally administered to human subjects in an amount of about 8 mg/kg (which approximately corresponds to an amount of 100 mg/kg in mice) per day, or once every two or three days. In one embodiment the composition of the invention is administered to human subjects intranasally in an amount of between about 0.5 mg/kg and 5 mg/kg (e.g., 0.8 mg/kg) daily or once every two or three days. In one embodiment the composition of the invention is administered to human subjects by inhalation in an amount of between about 0.05% and 10% (% of essential oil per inhalation volume) daily or once every two or three days. The compositions can be administered at any suitable time, e.g., prior to or after a meal, prior to activity, prior to sleeping and at different times of the day, e.g., in the morning, in the evening etc.

### EXAMPLES

### Example 1

### Preparation of the Pg Essential Oil

Plant material production: Pg varieties from the Newe Ya'ar living collection were vegetatively propagated (by rooting cut stems) and grown in experimental plots at the Newe Ya'ar Research Center. Fertigation was done by a dripping system.

Essential oil production: Steam-distillation was carried out in an essential oil pilot plant using a Clevenger apparatus system. 30 kg of fresh leaves and stems were distilled for 2.0 h in a 130 L container. After extraction, the oil was cooled and separated from the aqueous phase. The essential oil collected and stored in darkness at 4-6° C., to avoid rapid oxidation.

Essential oil analysis: Qualitative and quantitative analysis of the volatile compounds of essential oil was carried out using a gas chromatograph-mass spectrometer (GC/MS) (Agilent Technologies, Palo Alto, CA, USA) equipped with an auto-sampler Combi PAL (CTC Analytic, CH-zwingen) and a Rtx-5SIL MS cap. column (95% dimethyl/5% diphenyl polysiloxane, 30 m×0.25 mm i.d.×0.25 Pm film thickness). The identification of volatile compounds was based on the comparison of the mass spectra, performed with MSDChem software (Agilent), with NIST/WILEY libraries, and relative retention indexes.

The composition of two separate preparations of the Pg oil (designated ND-4100 and ND-4482) is shown in Table 1.

TABLE 1

| | ND-4100 | ND-4482 |
|---|---|---|
| | Pg oil | Pg oil |
| NAME | % | % |
| □-Citronellol | 29.0 | 22.5 |
| Linalool | 7.4 | 9.7 |
| Geraniol | 8.7 | 7.9 |
| Citronellyl formate | 9.2 | 7.1 |
| Iso-Menthone | 6.6 | 7.0 |
| 10-epi-□-Eudesmol | 7.5 | 4.1 |
| Geranyl formate | 2.5 | 2.4 |
| □-Terpineol | 0.9 | 1.8 |
| Germacrene D | 2.0 | 1.7 |
| □-Bourbonene | 1.4 | 1.6 |
| Geranyl tiglate | 1.5 | 1.4 |
| E-Citronellyl tiglate | 2.2 | 1.3 |
| Geranyl propanoate | 1.1 | 1.3 |
| Caryophyllene E | 0.9 | 1.3 |
| □-Cadinene | | 1.2 |
| Geranyl butanoate | 1.2 | 1.2 |
| Viridiflorene | 1.2 | 1.2 |
| □-Pinene | 0.6 | 1.2 |
| Phenyl ethyl tiglate | 1.3 | 1.1 |
| E-□-Ocimene | 0.3 | 1.0 |
| trans-Calamenene | | 0.9 |
| cis-Rose oxide | 1.5 | 0.9 |
| Menthone | 0.4 | 0.9 |
| □-Copaene | 0.8 | 0.7 |
| Limonene | 0.3 | 0.7 |
| trans-Muurola-3,5-diene | | 0.7 |
| Z-□-Ocimene | 0.2 | 0.6 |
| Terpinolene | 0.2 | 0.6 |
| Myrcene | 0.2 | 0.6 |
| Geranyl acetate | | 0.6 |
| Geranial | 0.5 | 0.5 |
| Citronellyl propanoate | 0.5 | 0.5 |
| 6,9-Guaiadiene | 0.4 | 0.5 |
| allo-Aromadendrene | 0.4 | 0.5 |
| Citronellyl acetate | 0.5 | 0.4 |
| trans-Rose oxide | 0.4 | 0.4 |
| trans-Cadina-1(6),4- | 0.4 | 0.4 |
| diene | | |
| □-Humulene | 0.2 | 0.4 |
| trans-Cadina-1,4-diene | | 0.4 |
| Neral | tr | 0.4 |
| □-Phellandrene | 0.1 | 0.3 |
| Hinesol | | 0.3 |
| □-Amorphene | | 0.2 |
| t-Cadinol | | 0.2 |
| □-Agarofuran | | 0.2 |
| □-Eudesmol | 0.2 | 0.2 |
| □-Muurolene | 0.1 | 0.2 |
| p-Cymene | 0.1 | 0.2 |
| □-Copaene | | 0.2 |
| □-Cubebene | | 0.2 |
| □-Phellandrene | | 0.2 |
| □-Elemene | | 0.2 |
| □-Selinene | | 0.2 |
| □-Muurolene | | 0.2 |
| □-Selinene | | 0.1 |
| □-Gurjunene | | 0.1 |
| □-Terpinene | | 0.1 |
| Myrcenol | | 0.1 |
| neo-Menthol | 0.1 | 0.1 |
| □-Calacorene | 0.1 | 0.1 |
| □-Terpinene | | 0.1 |
| 1-epi-Cubenol | | 0.1 |
| □-Ylangene | | 0.1 |
| p-Cymenene | | 0.1 |
| Citronellal | | 0.1 |
| 1,8-Cineole | | 0.1 |
| Citronellyl butanoate | 1.3 | |
| □-Cadinene | 1.1 | |
| Agarospiral | 0.3 | |
| □-Cadinene | 0.2 | |
| Camphene | | tr |
| cis-Dihydroagarofuran | | tr |
| Total | 95.9 | 93.6 |

### Example 2

Pg Essential Oil Enriched with Citronellyl Formate and/or Citronellol Enhances Cognitive Function in an Animal Model of Closed Head Injury (CHI)

### Animals and Ethical Statement

This study was approved by the Institutional Animal Ethics Committee of the Sheba Medical Center and complied with the guidelines of the National Research Council Guide for the Care and Use of Laboratory Animals (NIH approval no. OPRR-A5011-01,). Male C57BL/6JOlaHsd mice (8-10 weeks old) weighing 20-25 grams were purchased from Envigo, Israel, and used in all experiments. The mice were maintained under a controlled 12 hours (h) light/12 h dark cycle, with food and water provided ad libitum.

### Closed Head Injury (CHI) Model

Experimental CHI was induced by using a modified weight drop device (Chen et al [13]; Flierl et al [14]). Briefly, under 3% isoflurane anesthesia and supplementary oxygen (confirmed by the loss of response to pinch of paw), a midline longitudinal incision is performed, and the skull is exposed. A Teflon tipped cone (2 mm diameter) is placed upside down 2 mm lateral to the midline and 2 mm posterior to the bregma in the mid-coronal plane. The head is held in place and a 95 g weight is allowed to free-fall on the cone from a pre-established height, resulting in focal injury to the left hemisphere. The height of the free-fall weight is determined by the weight of the mouse and the desired CHI severity. In the present experiments, a free-fall of 6.5-7 cm was selected to induce a moderate neurological severity injury (NSS of 6-7 at 1 hr post injury). Sham mice were anesthetized and subjected to the same skin incision as described above, with no further trauma. After CHI and recovery from anesthesia (within ~2 mins) the mouse is oxygenated for 30 sec (via a mask) with 95:5 oxygen:CO2 and returned to its home-cage. At 1 h after CHI, the NSS is evaluated. Mice with a severity score of 9 or 10 were excluded from the study. Analgesia was achieved with dipyrone-500 mg in 250 ml drinking water provided after injury for id. Animals displaying apnea were monitored carefully and returned to their cage when adequate oxygenation was restored.
a. The mice were divided into different experimental groups each receiving a different treatment, as outlined below.

All experimental groups were followed up and subjected to the following tests:

### (i) Neurological Severity Score:

The functional status of the mice was evaluated according to the neurobehavioral score, known as the Neurological Severity Score (NSS), which consists of several tests that assess reflexes, alertness, coordination, and motor abilities. The score is a 10-point scale that is based on the presence of some reflexes and the ability to perform motor and behavioral tasks, such as beam walking, beam balance, and spontaneous locomotion (Beni-Adani et al., 2001 [15]; Flierl et al., 2009 [14]). Animals are awarded one point for failure to perform a task, the NSS thus increasing with the severity of dysfunction. The NSS obtained 1 hr after CHI reflects the initial severity of injury (Tsenter et al., 2008 [16]).

### (ii) Cognitive Evaluation, Including the Following Behavioral Tests:

### Barnes Maze (BM):

The BM test is based on the instinct of the mouse to hide when exposed to environmental disturbances and is used to test spatial learning and memory. The maze consists of an elevated circular platform with holes in the perimeter and a small dark hidden goal box. Bright light and aversive noise (85 dB) force the animals to escape from the open platform surface to find the hole under which the dark chamber (21×22×21 cm), “target goal box,” is located. Visual cues of different colors and shapes are placed around the room. The mouse was placed under a cylindrical black start chamber at the center of the maze. After 10 secs, the chamber was lifted, the buzzer was switched on and the mouse was allowed to explore the maze for 3 min. The trial ended when the mouse had reached the target box or after 3 min had elapsed. Immediately after entering the target box, the buzzer was turned off and the mouse was allowed to remain there for 1 min. Animals underwent 4 trials/day for 4 days, with an inter-trial interval of 15 min. A recall test (probe) is performed as follows: 24 h and seven days after the final session of acquisition training, mice undergo a 3 min probe trial in which the escape box is removed from the apparatus. The probe trial is performed in a similar manner to the acquisition trials. For the probe trial, the latency to reaching the escape box first is recorded.

The Barnes maze test was performed between days 14-17 (2 weeks two weeks following the injury) plus a recall test on days 18 and 25 after injury (data not presented), for a total of 3 rounds.

### Novel Object Recognition Test:

The novel object recognition (NOR test) was performed to assess short term memory 22d after trauma. On the first day of the test, mice were placed in the testing cage (a container measuring 60×25×40 cm) for 1 h habituation. On the following day they were returned to the same testing cage with two identical objects of similar size, surface complexity and material. The cumulative time spent by each mouse exploring the objects was recorded manually for a total of 5 min. After 4 hours each mouse was reintroduced into the cage, where one of the familiar objects was replaced with a new one. A normal mouse spends relatively more time exploring a novel object than a familiar one, attesting to its ability to remember and distinguish novel from familiar. Exploration of the object was determined as such when the mouse's nose was pointed in the direction of the object and the mouse was actively investigating it, directly touching it or being close to it. Toys were used as NOR objects (a toy car and a doll). The “old” and “new” objects were significantly different in shape, colors, texture, and material (plastic and ceramic) but not in size (about 5×5×5 cm per object) or surface complexity. The time spent exploring each of the objects (novel or familiar) was recorded and calculated as the percentage of time from a total of 5 min.

The NOR test was performed on all the experimental groups as described above, 22 days after TBI.

### Y Maze Test:

At 24 days post CHI, the Y maze spatial memory test was preformed to evaluate short term spatial memory. The maze is designed as three black Perspex arms at a 120° angle from one another (“start,” “other,” and “new” arms). A mouse is placed at the “start” arm and allowed to explore freely this arm and the “other” arm for 5 min, while the “new” arm remains closed. After 2 min the mouse is returned to the maze and allowed to explore all 3 arms for 2 min. The amount of time spent in each arm is documented. Short-term memory is reflected by the Preference index (PI), calculated as the amount of time spent in the new arm relative to the total amount of time spent in the “other” plus “new” arms. EthoVision XP10 software (Noldus) was used to evaluate the time spent in each arm.

### Results

Mice were subjected to a closed head injury (CHI), as described in Materials and methods above and were fed with Pg essential oil, citronellol, citronellyl formate or their combinations starting 24 hours after the trauma. The treatment included a total of 9 daily doses spread over 2 weeks. NSS evaluation to determine clinical, especially motor, function was performed 1 hour, 24 hours, 48 hours, 72 hours, 1 week, and two weeks after injury.

### The Experimental, Treatment Groups Included the Following:

Pg oil (100 mg/kg)
Citronellol (Sigma-Aldrich, Israel) (25 mg/kg)
Citronellyl formate (Ambeed, Arlington Hts, IL, USA) (80 mg/kg)
Pg oil (100 mg/kg)+citronellol (25 mg/kg)
Pg oil (100 mg/kg)+citronellol (25 mg/kg)+citronellyl formate (80 mg/kg)
Vehicle solution

In addition, the experiments also included a Sham group, namely, mice with no head injury and no treatment with vehicle.

Pg oil extract was diluted with olive oil to a final concentration of 13 mg/ml. Pg oil was freshly diluted each day during the treatment period in glass vials and was administered by oral gavage in an equivalent volume of 200 μl as the vehicle solution.

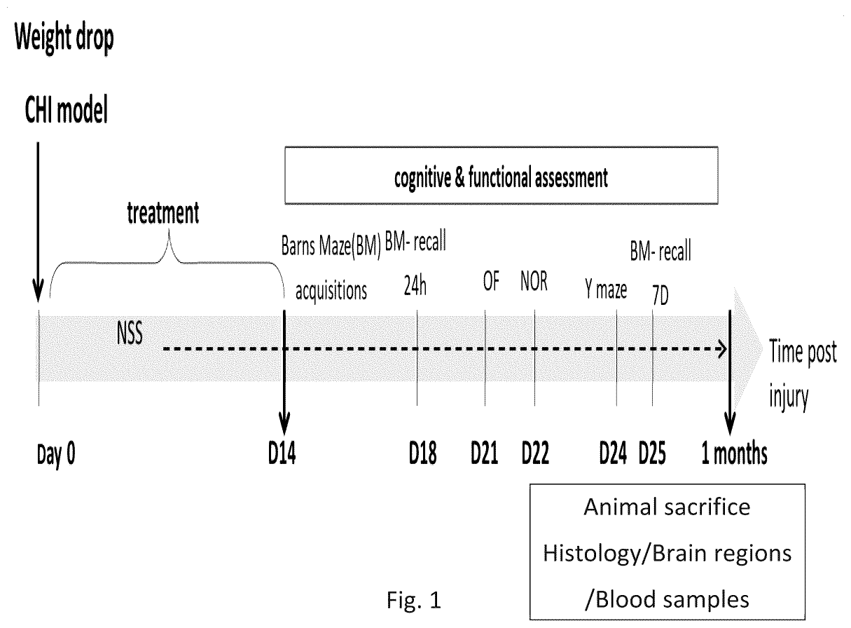

Behavioral tests were performed immediately after completion of the treatment period. An outline of the study design is shown in FIG. 1. The time point of the closed head injury (CHI) induction was defined as Day 0, followed by a treatment period of two weeks ending at D14. During this period, NSS was determined. After D14 commenced the cognitive and functional assessment of the mice in the various treatment groups: Barnes maze (BM) acquisitions took place at days D14-D17 (4 days). Open Field (OF) test, for assessing anxiety and natural exploration behavior, was performed at D21, the NOR test was performed at D22 after the trauma and the Y maze testing took place at D24 after the trauma.

### Neurological Severity Score (NSS)

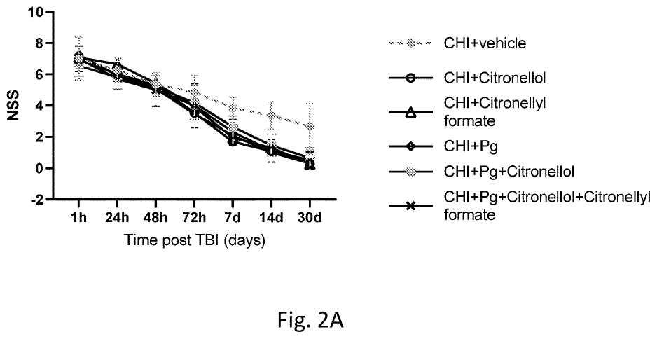

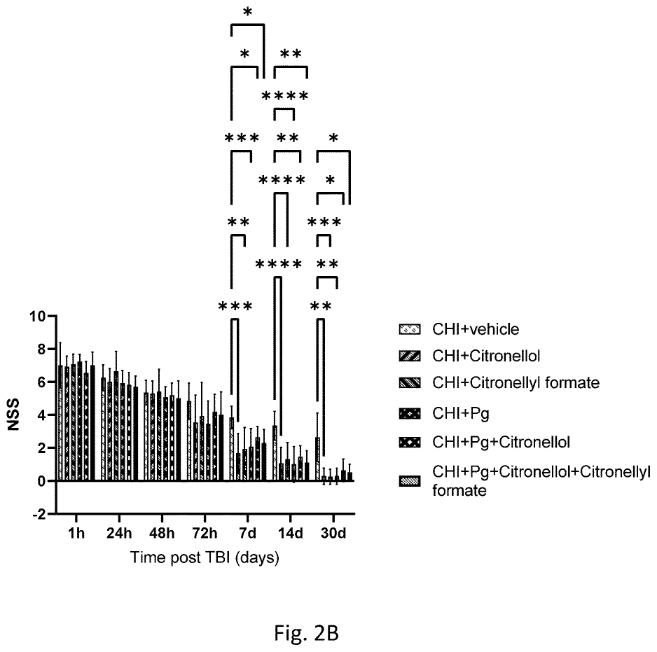

The neurological severity score (NSS) was determined for each of the experimental groups of mice (details as described above) starting 1 hour after the trauma. As can be seen in FIGS. 2A and 2B, starting from day 7 up to 30 days following the trauma (the last time point measured), a significant reduction in the NSS (which is an indication of improvement in motor functions) was observed in all the experimental groups as compared with the vehicle treated group of CHI mice.

This is a very interesting observation which emphasizes the unique effect of the combination treatment on the cognitive/behavioral functions of the traumatized mice, as shown in the cognitive tests described above.

### Barnes Maze Test—Learning

Following the treatment period (namely, two weeks after the trauma, and 3 days after the last treatment), the mice were subjected to testing in a Barnes maze, as described above. The test that is performed immediately after the acquisition stage measures the learning ability.

Interestingly, mice that were treated with citronellol alone, or citronellyl formate alone showed a significant improvement, but only on day 2.

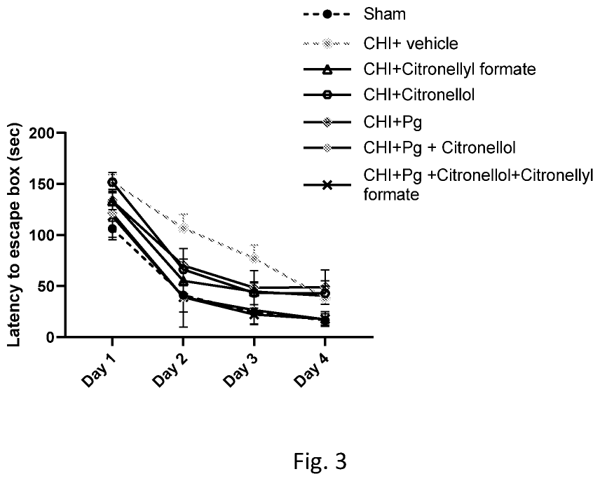

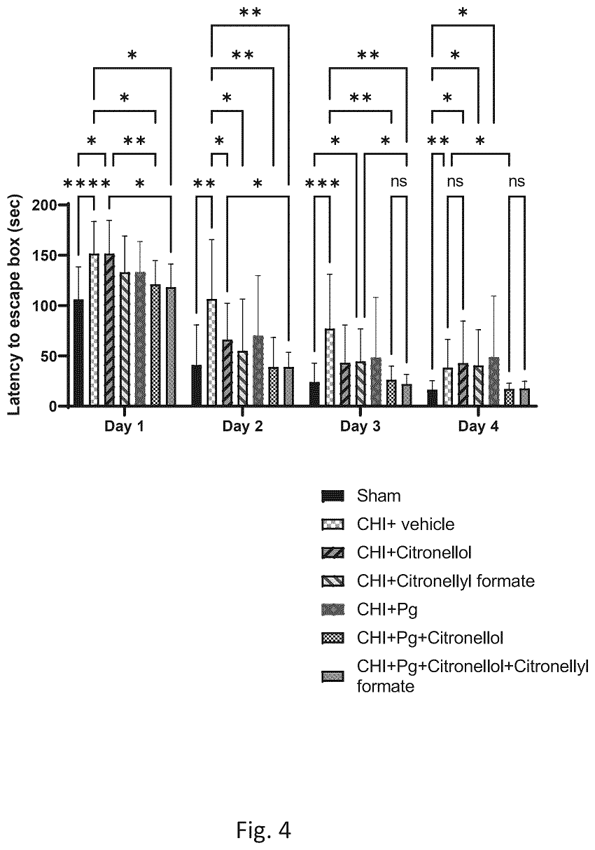

Only mice that were treated with enriched Pg oil (i.e., Pg oil at a suboptimal concentration of 100 mg/kg+citronellol (25 mg/kg), or Pg oil at a suboptimal amount of 100 mg/kg+citronellol (25 mg/kg)+cytronellyl formate (80 mg/kg)) performed as good as healthy mice that were not traumatized at all (sham) at days 1-4. Their performance was significantly better (namely, the mice learnt faster) than mice treated with vehicle alone or with any of the other treatments provided as a monotherapy (namely, Pg oil at a suboptimal amount, citronellol, or cytronellyl formate) (FIG. 3 and FIG. 4).

No significant difference was found between the two types of combinations.

### Novel Object Recognition (NOR) Test

All the experimental groups of mice were subjected to a Novel Object Recognition (NOR) test (details as described above) performed 22 days after trauma.

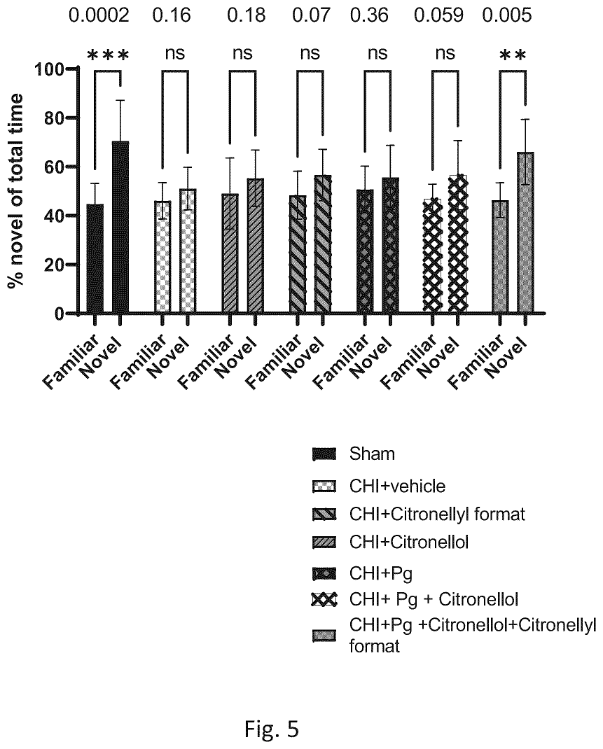

As can be seen in FIG. 5, sham mice were able to prefer/recognize a novel object when compared with a familiar one. In contrast, mice subjected to CHI were not able to prefer/recognize a novel object when compared with a familiar one.

Mice subjected to CHI and treated with either Pg oil alone, or citronellol or citronellyl formate were also not able to prefer/recognize a novel object when compared with a familiar one.

Mice subjected to CHI and treated with the combination of Pg oil+citronellol showed a trend towards a preference/recognition of a novel object when compared with a familiar one (p<0.059).

A significant improvement (p<0.05) was found in the memory capability of the mice treated with the enriched Pg oil, namely animals that were treated with the combination of Pg oil at a suboptimal amount+citronellol+cytronellyl formate, whereby 4 hours after the familiar object was replaced with a novel object, the percent of the time that the treated mice spent by the novel object was significantly higher than that spent by untreated mice (CHI+vehicle) which appeared not to have any memory of the familiar object (FIG. 5).

### Y Maze Test

All the experimental groups of mice were subjected to a Y maze test (details as described above) performed 24 days after trauma.

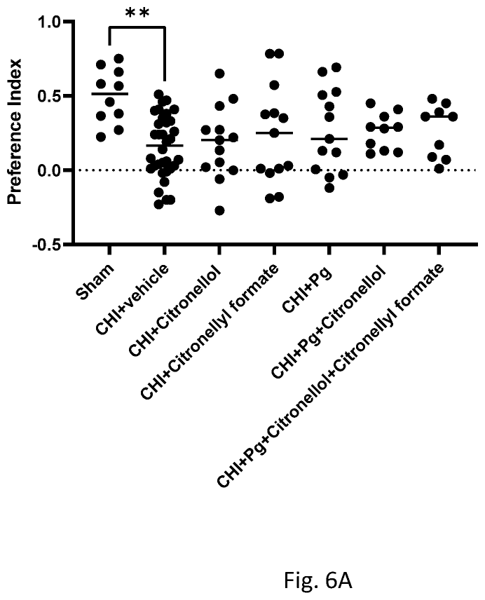

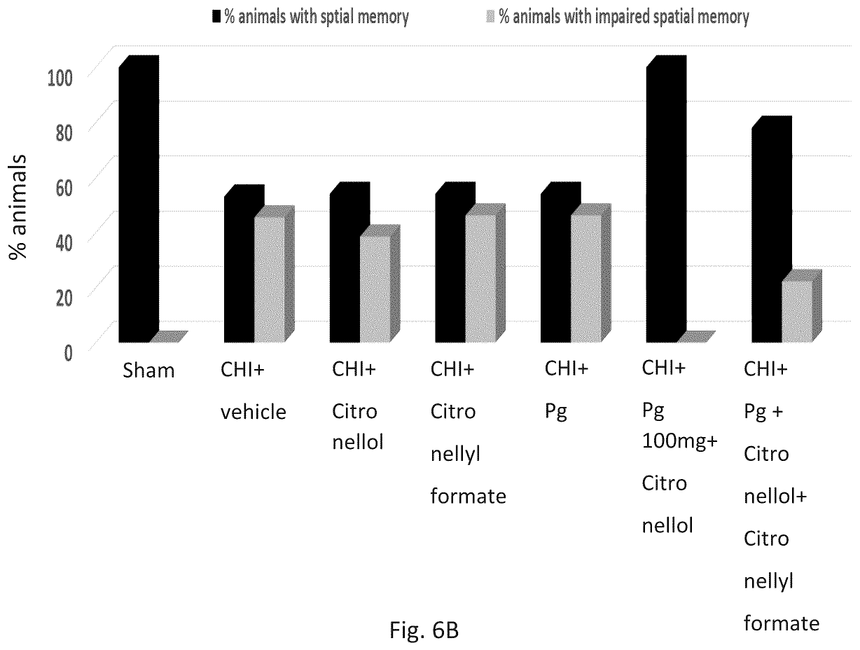

FIG. 6A shows the distribution of the PI values for each mouse in each of the experimental groups. The data was further analyzed by dividing the mice's performance into two groups: mice with (+) positive y-axis values (which represent a good spatial memory) and mice with negative (−) y-axis values (which represent spatial memory loss). The results are presented in FIG. 6B.

As can be seen in FIGS. 6A and 6B, sham animals performed significantly better compared to CHI-vehicle animals, and their PI (preference index) was significantly higher, which represents a better spatial memory. All sham animals (100%) preferred the novel arm (which is an indication of a good spatial memory) over the familiar arm.

Only 53% of CHI-vehicle mice preferred the novel arm.

Like the sham mice, 100% of mice treated with Pg oil+citronellol preferred the novel arm over the familiar arm.

78% of the mice treated with Pg oil+citronellol+Citronellyl-formate preferred the novel arm over the familiar arm.

About 54% of the mice treated with Pg oil or citronellol or Citronellyl formate alone preferred the novel arm over the familiar one.

To conclude, the behavioral tests revealed that traumatized mice treated with the Pg essential oil (suboptimal amount) combined with citronellol and optionally also with citronellyl formate, showed a significant improvement in learning and memory functions.

### Example 3

Administering Enriched Pg Essential Oil to Patients Suffering from TBI

Patients suffering from traumatic brain injury are treated with the enriched Pg oil of the invention, namely treated with a composition comprising a combination of Pg oil (optionally at a suboptimal amount)+citronellol, and optionally+citronellyl formate.

The treatment is by oral, subcutaneous injection, or nasal administration and is given immediately after the trauma followed by a 12-days treatment session in which the subject receives a total of 9 treatments, administered once a day or once in two or three days. The following dosages are administered: 1.25 or 2.5 mg/kg (for intranasal administration), and 2 mg/kg, 4 mg/kg, 5 mg/kg, 8 mg/kg, or 20 mg/kg (for oral administration). Optionally, additional treatment is given for two weeks, 9 months after the injury.

The patients undergo cognitive evaluation to assess efficacy of treatment.

1.-51. (canceled)

52. A pharmaceutical or nutritional composition for treating, preventing, or ameliorating traumatic brain injury in a subject or for improving cognitive function of a subject suffering from traumatic brain injury, the composition comprising:
(a) essential oil extracted from Pelargonium graveolens (Pg oil);
(b) at least one additional monoterpene or monoterpenoid, wherein the final concentration (weight/weight) of said at least one additional monoterpene or monoterpenoid is at least 50% higher than the concentration of said monoterpene or monoterpenoid in the Pg oil; and optionally
(c) one or more physiologically acceptable carriers.

53. The composition according to claim 52 wherein said at least one additional monoterpene or monoterpenoid is selected from a group consisting of citronellol, citronellyl formate, linalool, geraniol and menthone.

54. The composition according to claim 52 wherein said composition comprises a concentration of between about 30% and 80% citronellol, or a concentration of between about 10% and 80% citronellyl formate.

55. The composition of claim 52 wherein said composition is administered at a concentration of between about 0.5 mg/kg and 50 mg/kg.

56. The composition of claim 52 wherein said composition comprises less than 200 mg/kg essential oil extracted from Pelargonium graveolens.

57. The composition of claim 52 wherein said composition is administered once, twice, three times or more daily, or once every two days or every three days for a treatment session of between about 5 days and four weeks, or more following the TBI, or administered in a prophylactic manner to subjects at risk of TBI.

58. The composition of claim 52 wherein said composition is provided orally at a concentration of between about 5 mg/kg and about 50 mg/kg once a day or once every two or three days for a treatment session that comprises between about 8 and about 12 daily treatments per session, e.g., 9 daily treatments per session.

59. The composition of claim 52 wherein said composition is provided by intranasal administration at a concentration of between about 0.5 mg/kg and about 5 mg/kg once a day or once every two or three days for a treatment session that comprises between about 8 and about 12 daily treatments per session, e.g., 9 daily treatments per session.

60. The composition of claim 52 wherein said composition is provided by inhalation at a concentration of between about 0.1% and about 10% once a day or once every two or three days for a treatment session that comprises between about 8 and about 12 daily treatments per session, e.g., 9 daily treatments per session.

61. A method of treating, preventing, or ameliorating TBI, or for improving cognitive function of a subject suffering from TBI, the method comprising administering to a subject in need thereof a therapeutically effective amount of a composition comprising:
(a) essential oil extracted from Pelargonium graveolens (Pg oil);
(b) at least one additional monoterpene or monoterpenoid, wherein the final concentration in the composition (weight/weight) of said at least one additional monoterpene or monoterpenoid is at least 50% higher than the concentration of said monoterpene or monoterpenoid in the Pg oil; and optionally
(c) one or more physiologically acceptable carriers.

62. The method according to claim 61 wherein said at least one additional monoterpene or monoterpenoid is selected from a group consisting of citronellol, citronellyl formate, linalool, geraniol and menthone.

63. The method according to claim 61 wherein said composition comprises a concentration of between about 30% and 80% citronellol or a concentration of between about 10% and 80% citronellyl formate.

64. The method according to claim 61 wherein said composition is administered at a concentration of between about 0.5 mg/kg and 50 mg/kg.

65. The method according to claim 61 wherein said composition comprises less than 200 mg/kg essential oil extracted from Pelargonium graveolens.

66. The method according to claim 61 wherein said composition is administered once, twice, three times or more daily, or once every two days or every three days for a treatment session of between about 5 days and four weeks, or more following the TBI, or administered in a prophylactic manner to subjects at risk of TBI.

67. The method according to claim 61 wherein said composition is administered in a treatment session that comprises between about 8 and about 12 daily treatments, e.g., 9 daily treatments per session.

68. The method according to claim 61 wherein said composition is provided orally at a concentration of between about 5 mg/kg and about 50 mg/kg once a day or once every two or three days for a treatment session that comprises between about 8 and about 12 daily treatments per session, e.g., 9 daily treatments per session.

69. The method according to claim 61 wherein said composition is provided by intranasal administration at a concentration of between about 1 mg/kg and about 5 mg/kg once a day or once every two or three days for a treatment session that comprises between about 8 and about 12 daily treatments per session, e.g., 9 daily treatments per session.

70. The method according to claim 61 wherein said composition is provided by inhalation at a concentration of between about 0.05% and about 10% once a day or once every two or three days for a treatment session that comprises between about 8 and about 12 daily treatments per session, e.g., 9 daily treatments per session.

71. A pharmaceutical or nutritional composition for treating, preventing, or ameliorating traumatic brain injury in a subject, the composition comprising:
(a) essential oil extracted from Pelargonium graveolens (Pg oil);
(b) at least one exogenous monoterpene or monoterpenoid; and optionally
(c) one or more physiologically acceptable carriers.
